(19) United States
(12) Patent Application Publication
(10) Pub. No.: US 2005/0033421 A1
(43) Pub. Date: Feb. 10, 2005

(54) Intraocular lens

(76) Inventor: Peter Toop, Bucks (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/633,209

(22) Filed: Jul. 31, 2003

(51) Int. Cl. A61F 2/16

(52) U.S. Cl. 623/6.11

(57) ABSTRACT

An intraocular lens comprising a toric optic and one or more haptics, wherein the thickness of a region of the distal part of the, or each, haptic is greater than the rest of the haptic, such that rotation of the lens is inhibited in use.

### FIELD OF THE INVENTION

This invention relates to an intraocular lens comprising a toric optic.

### BACKGROUND OF THE INVENTION

A typical intraocular lens comprises an optic and two opposed haptics. Conventional intraocular lenses are often prone to “haptic failure”, wherein excessive capsular contraction causes the haptics to buckle or twist, resulting in their dislocation. The haptics should maintain the optic centrally in the capsular sac and should not be so springy as to damage the sac after insertion.

EP-A-0962196 describes an intraocular lens wherein the haptics are shaped such that, in a first stage of compression, the proximal part of the haptic can be fully compressed; and in a second stage, the distal part of the haptic can be compressed, to provide a lens that is essentially resistant to haptic failure.

Astigmatism is a visual defect that can arise from an irregularity in the shape of the cornea. An astigmatic cornea is defined by spherical and cylindrical axes, the vertical meridian providing a different optical power to that of the horizontal meridian. The unequal curvature of the cornea causes light entering the eye to focus at different points, thus distorting vision. Astigmatism is often coupled with other visual impairments such as myopia and hypermetropia.

Astigmatism can be corrected by using a toric lens. A toric lens is designed to match the unequal curvatures of an astigmatic cornea. One of the requirements of a toric lens is that the optic must remain correctly aligned along the spherical and cylindrical axes. Thus, whereas spherical lenses may rotate freely, toric lenses require a means with which to inhibit rotation of the lens. In contact lenses, this is often accomplished by slightly weighting the lens, e.g. by making one or more sections of the lens periphery thicker (or thinner) than other sections.

### SUMMARY OF THE INVENTION

According to an aspect of the invention, an intraocular lens comprises a toric optic and one or more haptics. A region of the distal part of the or each haptic is thicker than the rest of the haptic. The thicker region of the distal part is preferably a peripheral region.

Preferably, the or each haptic is compressible, in the plane of the lens. More preferably, the or each haptic is curved, and shaped such that, in a first stage of compression, the proximal part of the haptic can be fully compressed and, in a second stage, the distal part of the haptic can be compressed.

The or each haptic may include an aperture, of which opposed points are brought into contact in the first stage of compression. The or each stage of compression may be essentially continuous, full compression being reached gradually from the proximal end towards the distal end of the haptic. A lens of the invention preferably comprises two or more haptics, compression of the haptics preferably providing an essentially elliptical form of the lens.

In use, the thicker distal region(s) of the haptic(s), nestles into the wall of the capsular sac, inhibiting rotation of the lens. This allows the toric optic to remain correctly aligned along the spherical and cylindrical axes. A compressible haptic may further stabilise the lens during use.

### BRIEF DESCRIPTION OF THE DRAWINGS

In the accompanying drawings (which are given by way of example only):

FIGS. 1A and 1B are respectively plan and side views of an intraocular lens embodying the present invention. FIG. 1C depicts the same lens in a capsular sac.

FIG. 2 is a comparative diagram showing the effect of capsule sac size on a lens embodying the present invention and two conventional toric lenses.

### DESCRIPTION OF THE PREFERRED EMBODIMENTS

Embodiments of the invention will now be described by way of example only, with reference to the accompanying drawings.

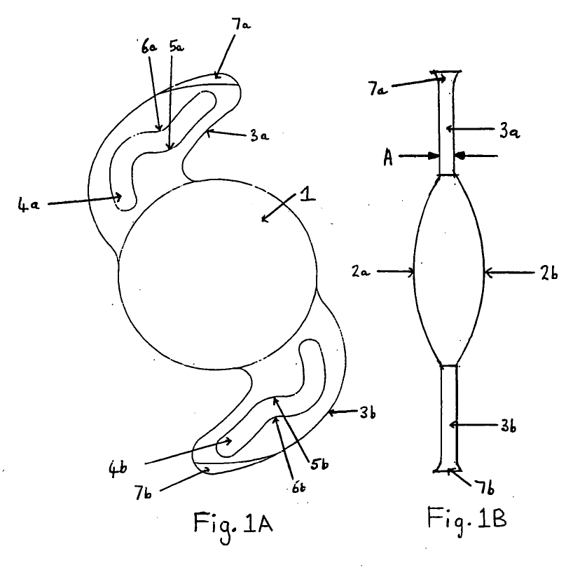

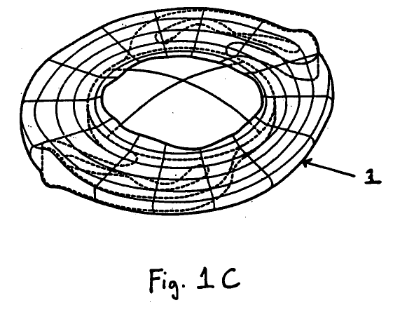

FIGS. 1A and 1B show an intraocular lens having a toric optic 1, comprising convex faces 2a and 2b. The lens comprises haptics 3a and 3b, each joined to the optic. Each haptic comprises an aperture, 4a and 4b. Opposed points of each haptic 5a and 6a, and 5b and 6b, are shown.

These features are such that initial compression of the haptic leads to abutment of opposite walls of the aperture, bringing the opposed points into contact, thereby defining a proximal part that is fully compressed and a distal part that can undergo further compression. Such further compression brings the distal end of each haptic substantially into contact with the periphery of the optic, to give an essentially elliptical shape, in plan. These features allow restricted rotation of the lens.

FIGS. 1A and 1B also show the thicker peripheral regions 7a and 7b of the distal part of each haptic; haptic thickness is denoted by the dimension A shown in FIG. 1B. These thicker regions inhibit rotation of the lens when in use, thus stabilising the optic along the spherical and cylindrical axes. FIG. 1C shows a capsular sac 1 surrounding the same lens (shown by dashed lines). The thicker distal regions nestle in the wall of the sac, inhibiting rotation of the lens.

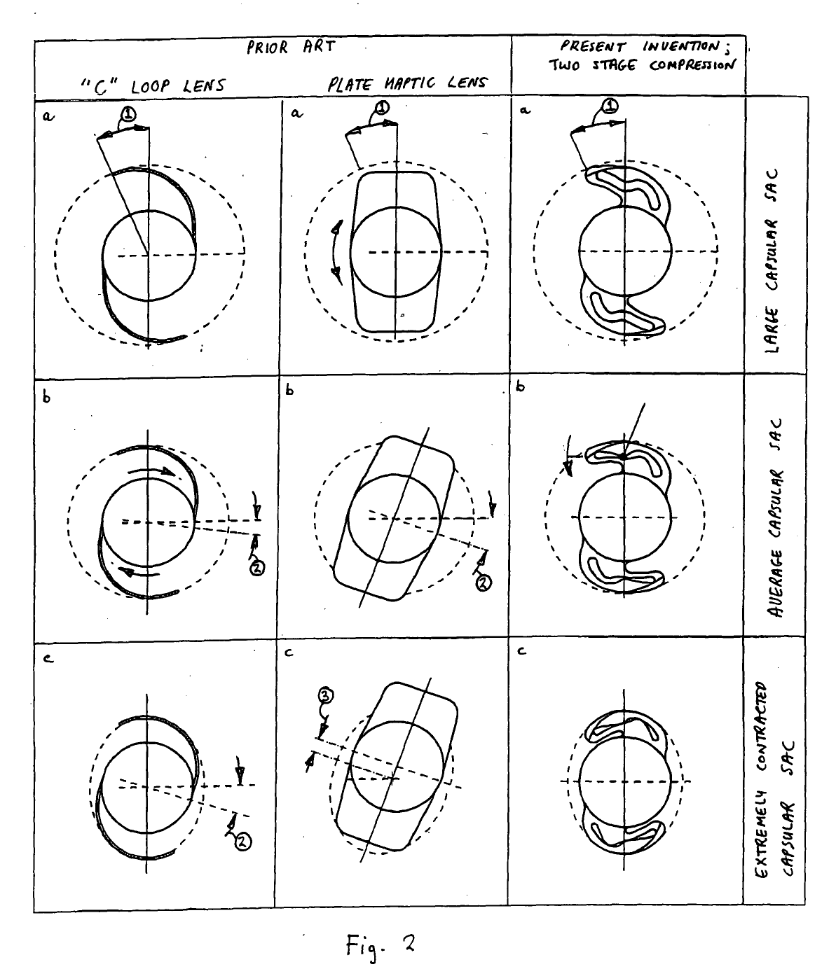

FIG. 2 shows the effect of capsular sac size for a variety of intraocular lenses. The left column depicts a known “C-loop” lens, the middle column a known “plate-haptic” lens and the right column a lens embodying the present invention. Rows a, b and c, correspond to large, average and extremely contracted capsular sacs respectively. The intended angular position, rotational misalignment and translational misalignment are shown by labels 1, 2 and 3 respectively.

In each example of the “C-loop” lens, the optic of the lens can rotate, causing the toric optic to misalign. Rotation and thus misalignment becomes more severe as the capsular size decreases.

In a large capsular sac, the “plate-haptic” lens can rotate to an unpredictable position. When the capsule contracts to an average size, the haptic can fixate, but the lens often becomes rotationally misaligned. In the case of the severely contracted capsule, the lens may undergo translation causing the haptics to rupture the wall of the sac.

In each example of a lens of the invention, the thicker distal regions of the haptics nestle into the wall of the sac, inhibiting rotation. For the large sac, there is little haptic compression but the thicker distal regions still restrict rotation. For the average sac, the semi-compressed haptics inhibit rotation, with the thicker regions further stabilising the lens. Rotation is similarly inhibited in the contracted sac, except that the haptics are now fully compressed.

1. An intraocular lens comprising a toric optic and one or more haptics, wherein the thickness of a region of the distal part of the, or each, haptic is greater than the rest of the haptic, such that rotation of the lens is inhibited in use.

2. The lens according to claim 1, wherein the thickness of the, or each, haptic is greatest at the periphery.

3. The lens according to claim 1, wherein the, or each, haptic is compressible, in the plane of the lens.

4. The lens according to claim 3, wherein the, or each, haptic is curved, and shaped such that, in a first stage of compression, the proximal part of the haptic can be fully compressed and, in a second stage, the distal part of the haptic can be compressed.

5. The lens according to claim 4, wherein the, or each, haptic includes an aperture of which opposed points are brought into contact, in the first stage of compression.

6. The lens according to claim 4, wherein the, or each, stage of compression is essentially continuous, full compression being reached gradually from the proximal end towards the distal end of the haptic.

7. The lens according to claim 4, which comprises two or more haptics, wherein the haptics are compressed to provide an essentially elliptical form of the lens.
